(19) United States
(12) Patent Application Publication
(10) Pub. No.: US 2013/0072808 A1
(43) Pub. Date: Mar. 21, 2013

(54) STRUCTURED PROBES FOR NEURAL APPLICATIONS

(71) Applicants: Hercules Pereira Neves, Hamme-Mille (BE); Patrick Ruther, Karlsruhe (DE); Stanislav Herwik, Rheinfelden (DE); Tom Torfs, Kraainem (BE)

(72) Inventors: Hercules Pereira Neves, Hamme-Mille (BE); Patrick Ruther, Karlsruhe (DE); Stanislav Herwik, Rheinfelden (DE); Tom Torfs, Kraainem (BE)

(73) Assignees: IMTEK, Freiburg (DE); IMEC, Leuven (BE)

(21) Appl. No.: 13/234,773

(22) Filed: Sep. 16, 2011

(51) Int. Cl. A61B 5/0484; H01R 43/16

(52) U.S. Cl. 600/544; 29/874

(57) ABSTRACT

A probe for recording and/or stimulating brain activity includes a connecting portion and at least one shank extending from the connecting portion. The at least one shank includes a first side, a second side opposed to the first side, and a fin protruding substantially perpendicularly from the second side and running on at least a part of a length of the at least one shank. The first side includes at least one recording and/or stimulating site.

### BACKGROUND

Unless otherwise indicated herein, the materials described in this section are not prior art to the claims in this application and are not admitted to be prior art by inclusion in this section.

The recording or stimulation of neurons helps understanding how the brain processes information and controls bodily functions. In order to enable the recording or stimulation of the brain, a neural microelectrode (also called neural probe) can be inserted into a brain until the tip reaches the region of the brain to be stimulated/recorded.

A major criterion for the neural microelectrode design is to have as minimal a footprint as possible to minimize damage to the brain tissue and neuron network. Typically, a shank thickness of less than 50 μm for silicon-based probes is preferred. When multisite elongated neural microelectrode structures (e.g. with lengths longer than 6 mm) are desired, the design and fabrication of the microelectrodes is further complicated. The electrodes should have the ability to withstand the different types of forces during the insertion phase of the surgical implantation of the probe. The main forces that act upon the electrode probe during handling and insertion into the brain tissue are: the bending force which is a result of the out-of-plane loading causing parallel displacement to the tissue plane, the buckling force which occurs under axial compression as a result of the force that counteracts the normal force exerted on the brain tissue by the probe, and the shear force that prevents the tip of the probe from slipping on the surface of the brain tissue.

As one wants to make the neural probes as thin as possible the fragility of the probe also increases, particularly when relatively brittle materials such as silicon are used.

US2009012593 discloses a multiple-electrode probe for deep brain stimulation whereby reinforcements are used. The neural probe comprises longitudinal beam structures made of a biocompatible metal or carbon fiber. U.S. Pat. No. 5,855,801 discloses a micro needle fabricated by using heavy boron doping to define the shape of the probe cross-section. The probe is sequentially reinforced by thickening the probe by leaving undoped silicon on the probe. Such a heavy boron doping is typically incompatible with the integration of electronics on the shaft itself.

A need still exists for probes for brain recording or stimulation which can pierce through the brain membrane (also called Dura matter) without causing harmful dimpling of the Dura (e.g. which comprise a small cross-section) whilst providing a solution for the problem of bending and buckling. There is also still a need for a method for fabricating the same.

### SUMMARY

It is an object of the present disclosure to provide a probe for brain recording or stimulation and a method for fabricating the same.

This object is met with the means according to the independent claims of the present disclosure. The dependent claims relate to preferred embodiments.

In a first aspect, the present disclosure relates to a method for manufacturing a probe for brain recording and/or stimulation, the probe comprising one or more shanks having a same orientation, wherein each of the shanks has a first side and a second side opposed to the first side and comprises at least one recording and/or stimulating site on the first side and a fin (or fins) protruding perpendicularly from the second side and running on at least a part of the length of the shank along the same orientation.

In an embodiment, the method may comprise the steps of: providing a substrate having a first surface and a second surface opposed to the first surface, the first surface comprising the at least one recording and/or stimulating site of each of the one or more shanks of the probe to be manufactured, and removing part of the substrate at the second side or attaching a fin (or fins) to the second side so as to form a substrate having a fin (or fins) protruding perpendicularly from the second side and running on at least a part of the length of the shank to be manufactured along the same orientation. In particular, the method may comprise the steps of:

a1) providing a substrate having a first and a second surface opposed to the first surface, the first surface comprising the at least one recording and/or stimulating site of each of the one or more shanks of the probe to be manufactured,

a2(i) providing a first etch mask pattern on the second surface, the first etch mask pattern masking the area corresponding to the fin of each of the one or more shanks of the probe to be manufactured, the first etch mask pattern being resistant to an etching process, and

a2(ii) etching the second surface with a etching process for which the first etch mask is resistant, in a direction perpendicular to the second surface in such a way as thinning, but not eliminating, at least part of the substrate away from the first etch mask pattern, thereby defining the height of the fins. As used herein and unless provided otherwise, the term “etching” encompass both dry-etching and wet-etching but dry-etching is advantageous due to its anisotropic character. Wet-etching can be made anisotropic e.g. by using an Si substrate.

The method of the first aspect is advantageous as it permits the production of thin shanks having a desirable rigidity in the direction perpendicular to the surface of their first or second side which is similar to the rigidity of thicker shanks. This rigidity is about the same as the rigidity of a shank not having the fin but having a thickness equal to the sum of the thickness of the shank and the height of the fin. The shanks of the present disclosure are therefore rigid enough to penetrate the brain without bending while being thin enough to minimize damage to the brain.

Preferably, the method provides the shank(s) with a T-shaped cross-section.

In a preferred embodiment of the first aspect, the present disclosure relates to a method for manufacturing a probe for brain recording and/or stimulation, the probe comprising a connector part from which one or more shanks protrude along a same orientation, wherein each of the shanks has a first side and a second side opposed to the first side and comprises at least one recording and/or stimulating site on the first side and a fin (or fins) protruding perpendicularly from the second side and running on at least a part of the length of the shank along the same orientation, thereby preferably providing the shank with a T-shaped cross-section, the method comprising the steps of:

a1. providing a substrate having a first and a second surface opposed to the first surface, the first surface comprising the at least one recording and/or stimulating site of each of the one or more shanks of the probe to be manufactured,

S. providing on the second surface, a second etch mask pattern delimiting the shape of the shanks and masking the area corresponding to the connector part of the probe to be manufactured, the second etch mask pattern being resistant to an etching process,

a2(i). providing a first etch mask pattern on the second surface, the first etch mask pattern masking exactly the area corresponding to the fins) of each of the one or more shanks and the area corresponding to the connector part of the probe to be manufactured by (preferably exactly) covering the second etch mask pattern, the first etch mask pattern being resistant to an etching process,

a2(ii). etching the second surface with an etching process for which the first etch mask pattern is resistant, in a direction perpendicular to the second surface in such a way as thinning, but not eliminating, at least part of the substrate away from the first etch mask pattern, thereby defining the height of the fin(s),

a2(iii). removing the first etch mask pattern without removing the second etch mask pattern,

a2(iv). etching the second surface with an etching process for which the second etch mask pattern is resistant, in a direction perpendicular to the second surface in such a way as thinning but not eliminating at least part of the substrate away from the second etch mask pattern, thereby defining the thickness of the shanks, and

a3. at least partially cutting out the probe from the substrate.

In a second aspect, the present disclosure relates to a probe for brain recording and/or stimulation, the probe comprising one or more shanks having a same orientation, wherein each of the shanks has a first side and a second side opposed to the first side and comprises at least one recording and/or stimulating site on the first side and a fin (or fins) protruding perpendicularly from the second side and running on at least a part of the length of the shank along the same orientation, preferably thereby providing the shank with a T-shaped cross-section.

In other words, the second aspect of the present disclosure may relate to a probe for brain recording or stimulation, the probe comprising shanks having a T-shaped profile, the T-shape being defined by a horizontal part and a vertical part, wherein the horizontal part has a first side and a second side, wherein the vertical part is attached to the second side, and wherein the first surface comprises one or more stimulating and/or recording sites (e.g. electrodes).

An advantage of embodiments of the present disclosure is that the second moment of inertia of the shanks of the probe is increased while maintaining a total cross-sectional area as small as possible.

In another embodiment, the present disclosure may relate to a probe for brain recording and/or stimulation, the probe comprising one or more shanks having a same orientation, wherein each of the shanks has a first side and a second side opposed to the first side and comprises at least one recording and/or stimulating site on the first side and a fin protruding perpendicularly from the second side and running on at least a part of the length of the shank along the same orientation, each of the shanks having a sharp pointed tip at its distal end, the tip lying in the plane defined by the first side.

In another embodiment, the probe may comprise at least one shank being a first shank according to any embodiment of the second aspect, which fin is further attached to either:

a) the fin of a second shank as described in any embodiment of the first aspect, or

b) the second side of a second shank having a first side and a second side opposed to the first side and comprising at least one recording and/or stimulating site on the first side, or

c) the second side of a second shank having a first side and a second side opposed to the first side,

thereby providing a probe having two parallel sets of shanks, wherein each shank in the first set is linked to a shank in the second set via the fin. In an embodiment, this further attachment of the fin may provide a H-shaped profile to the shank. In an embodiment, the fin of the second shank may belong to a second probe for brain recording and/or stimulation, comprising one or more shanks having the same orientation.

Such an H-shaped profile further rigidifies the shank and provides (in cases (a) and (b) two surfaces for recording and/or stimulating. Such an H-shaped shank is easier to insert in a brain than a flat shank of total thickness lower than the total thickness of the H-shaped profile. The reason is that in the H-shaped profile, each of the two shanks composing the H-shape can be thinner than the flat shank, even if the total structure is thicker. The fin being relatively thin, it does not interfere much with the penetration of the H-shaped shank. In an embodiment, the distance between two adjacent shanks in the first set is equal to the distance between two adjacent shanks in the second set.

In a third aspect, the present disclosure relates to a method of fabricating a probe for brain recording or stimulation, the probe having two parallel sets of shanks (e.g. comprising shanks having a H-shaped cross-section), the method comprising the step of attaching the free extremity of the fin of a shank of a probe according to any embodiment of the second aspect to either:

a) the fin of a second shank according to any embodiment of the second aspect, or

b) the second side of a second shank having a first side and a second side opposed to the first side and comprising at least one recording and/or stimulating site on the first side, or

c) the second side of a second shank having a first side and a second side opposed to the first side.

In an embodiment, the fin of the second shank may belong to a second probe for brain recording and/or stimulation, comprising one or more shanks having the same orientation.

In a fourth aspect, the present disclosure relates to a probe comprising one or more shanks having a same orientation, wherein each of the shanks has a first side and a second side opposed to the first side and comprises at least one recording and/or stimulating site on the first side which second side is attached to the second side of a second shank having a first side and a second side opposed to the first side and comprising at least one recording and/or stimulating site on the first side, thereby providing a probe having two parallel sets of shanks, wherein each shank in the first set is linked to a shank in the second set. In an embodiment, the distance between two adjacent shanks in the first set is equal to the distance between two adjacent shanks in the second set.

Particular and preferred aspects of the disclosure are set out in the accompanying independent and dependent claims. Features from the dependent claims may be combined with features of the independent claims and with features of other dependent claims as appropriate and not merely as explicitly set out in the claims.

Although there has been constant improvement, change and evolution of devices in this field and methods for fabricating the same, the present concepts are believed to represent substantial new and novel improvements, including departures from prior practices, resulting in the provision of more efficient, stable and reliable devices of this nature.

The above and other characteristics, features and advantages of the present disclosure will become apparent from the following detailed description, taken in conjunction with the accompanying drawings, which illustrate, by way of example, the principles of the disclosure. This description is given for the sake of example only, without limiting the scope of the disclosure. The reference figures quoted below refer to the attached drawings.

### BRIEF DESCRIPTION OF THE DRAWINGS

Further features of the present disclosure will become apparent from the drawings, wherein:

FIG. 1 is a schematic representation of a probe according to an embodiment of a second aspect of the present disclosure;

FIGS. 2A-2G illustrate a schematic representation of a method according to an embodiment of the first aspect of the present disclosure. In this representation, a cross-section orthogonal to the probe shank through one electrode is represented;

FIGS. 3A-3G illustrate a schematic representation of a method according to an embodiment of the first aspect of the present disclosure. In this representation, a cross-section along a shank is represented. The fin fabrication is not represented in this figure;

FIG. 4 illustrates a probe according to an embodiment of the present disclosure and showing the second side (backside) of the probe;

FIG. 5 illustrates of a probe according to an embodiment of the present disclosure and showing the first side (front side) of the shanks;

FIG. 6 illustrates the second side of the distal end of a shank in a probe according to an embodiment of the present disclosure;

FIG. 7 illustrates the first side of the distal end of a shank in a probe according to an embodiment of the present disclosure;

FIG. 8 is a schematic representation of a probe according to an embodiment of the second aspect of the present disclosure. A sharpened shank is represented but the fin is not shown;

FIG. 9A is a schematic perspective view of a shank having a T-shaped cross-section according to an embodiment of the present disclosure;

FIG. 9B is a schematic perspective view of a shank having a H-shaped cross-section according to an embodiment of the present disclosure;

FIG. 10 illustrates a probe array obtainable by an embodiment of the disclosure. The fins are not represented;

FIG. 11 shows a carrier block usable in the method of the disclosure, and an attachment location of a probe to the carrier block;

FIG. 12 shows a probe attached to a carrier block;

FIG. 13 shows a rotating grinding tool being used in conjunction with the carrier block and a probe attached thereto;

FIG. 14 shows the shape of the shanks distal ends before and after grinding, according to one embodiment of a method of the disclosure;

FIG. 15 shows the shape of the shanks distal ends before and after grinding, according to another embodiment of a method of the disclosure; and

FIG. 16 shows an example of the dimensions of a carrier block usable in a method of the disclosure.

### DETAILED DESCRIPTION OF EMBODIMENTS

The present disclosure will be described with respect to particular embodiments and with reference to certain drawings but the disclosure is not limited thereto but only by the claims. The drawings described are only schematic and are non-limiting. In the drawings, the size of some of the elements may be exaggerated and not drawn to scale for illustrative purposes.

Where an indefinite or definite article is used when referring to a singular noun e.g. “a” or “an”, “the”, this includes a plural of that noun unless something else is specifically stated.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present disclosure, the only relevant components of the device are A and B.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the disclosure described herein are capable of operation in other orientations than described or illustrated herein.

Reference throughout this specification to “one embodiment” or “an embodiment” means that a particular feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

In the drawings, like reference numerals indicate like features; and, a reference numeral appearing in more than one figure refers to the same element.

In a first aspect, the present disclosure relates to a method for manufacturing a probe for recording and/or stimulating brain activity, the probe comprising one or more shanks having a same orientation, wherein each of the shanks has a first side and a second side opposed to the first side and comprises at least one recording and/or stimulating site on the first side and a fin protruding perpendicularly from the second side and running on at least a part of the length of the shank along the same orientation, preferably thereby providing the shank with a T-shaped cross-section.

In an embodiment, the method may comprise the steps of:

a1) providing a substrate having a first surface and a second surface opposed to the first surface, the first surface comprising the at least one recording and/or stimulating site of each of the one or more shanks of the probe to be manufactured, and

a2) removing part of the substrate at the second side or attaching a fin to the second side so as to form a substrate having a fin protruding perpendicularly from the second side and running on at least a part of the length of the shank to be manufactured along the same orientation.

In another embodiment, the method may further comprise the step (a3) of cutting out at least partially the probe from the substrate and the step (b) of sharpening the distal end of the shanks in such a way as to provide the shanks with a sharp pointed tip at their distal end, the tip lying in the plane defined by the first side.

In another embodiment, the method may comprise the steps of:

a) providing a probe for brain recording and/or stimulation, the probe comprising one or more shanks having a same orientation, wherein each of the shanks has a first side and a second side opposed to the first side and comprises at least one recording and/or stimulating site on the first side and a fin protruding perpendicularly from the second side and running on at least a part of the length of the shank along the same orientation, and

b) sharpening the distal end of the shanks in such a way as to provide the shanks with a sharp pointed tip at its distal end, the tip lying in the plane defined by the first side.

In an embodiment, the sharpening may comprise the steps of

b1) providing a carrier having a first and second flat outer surface oriented at an angle to each other, so that the surfaces are joined by an edge of the carrier,

b2) attaching the shanks to the second surface of the carrier, with the second side of the shanks facing the carrier, the distal end being in close proximity to the edge, and

b3) subjecting the first surface of the carrier and at least part of the distal end of the second side of the probe to a grinding step, to thereby remove material at least from the distal end of the probe.

In step (b2), the shanks may be attached via their fins or via other means. In an embodiment, a major portion of the shanks may be attached (e.g. via a major portion of the fins) to the second surface. A major portion may mean more than 50% of the length, preferably more than 90% of the length.

In another embodiment, step (a2) may comprise the steps of

i) providing a first etch mask pattern on the second surface, the first etch mask pattern masking the area corresponding to the fin of each of the one or more shanks of the probe to be manufactured, the first etch mask pattern being resistant to an etching process, and

ii) etching the second surface with an etching process for which the first etch mask pattern is resistant, in a direction perpendicular to the second surface in such a way as thinning, but not eliminating, at least part of the substrate away from the first etch mask pattern, thereby defining the height of the fins.

Therefore, in an embodiment of the present disclosure, the method (or step (a) of the method) may comprise the steps of:

a1) providing a substrate having a first and a second surface opposed to the first surface, the first surface comprising the at least one recording and/or stimulating site of each of the one or more shanks of the probe to be manufactured,

a2(i)) providing a first etch mask pattern on the second surface, the first etch mask pattern masking the area corresponding to the fin of each of the one or more shanks of the probe to be manufactured, the first etch mask pattern being resistant to an etching process, and

a2(ii)) etching the second surface with an etching process for which the first etch mask is resistant, in a direction perpendicular to the second surface in such a way as thinning, but not eliminating, at least part of the substrate away from the first etch mask pattern, thereby defining the height of the fins.

This is advantageous as it permit the production of thin shanks having a rigidity in the direction perpendicular to the surface of their first or second side which is similar to the rigidity of thicker shanks. This rigidity is about the same as the rigidity of a shank not having the fin but having a thickness equal to the sum of the thickness of the shank according to the present embodiment and the height of the fin. The shanks of the present disclosure are therefore rigid enough to penetrate the brain without bending while being thin enough to minimize damage to the brain.

Step (a1) may involve providing a naked substrate, optionally providing a mask layer on this substrate, providing a (patterned) metal layer on the mask and passivating the metal layer, thereby defining the stimulating/recording sites and the corresponding leads.

The metal layer can for instance be deposited by physical vapor deposition, electron beam physical vapor deposition, thermo-evaporation, sputtering or electroplating amongst others. If the electrodes/sites are in IrO, electroplating can be used to grow IrO on Pt or sputtering can be used in an oxygen containing atmosphere.

In an embodiment of the first (and the second) aspect, the probe may further comprise a connector part from which the one or more shanks protrude along the same orientation. In this embodiment of the first aspect, in step (a2(i)), the first etch mask pattern may further mask the area corresponding to the connector part of the probe to be manufactured. This is advantageous because it prevents the connector to be thinned when the shanks are thinned and the height of the fins is defined.

In an embodiment of the first aspect, the first etch mask pattern may further be delimiting the shape of the shanks of the probe to be fabricated. This permits to thin the part of the substrate corresponding to the shanks to be manufactured, thereby defining the shanks.

In an embodiment, step (a2(i)) may comprise providing on the second surface, a layer covering the orthogonal projection on the second surface of the shape of the probe to be fabricated, the layer being resistant to the etching process, and patterning the layer.

The layer deposited in step (a2(i)) may for instance be deposited by chemical vapor deposition.

In an embodiment of the first aspect and of the second aspect, the fin may be running in the middle of the second surface of the shank so as to divide longitudinally the second surface in two equal parts. This geometry maximizes the rigidity of the shanks.

In an embodiment, the fin may be running on at least 50%, preferably at least 75%, more preferably at least 85% of the length of the shank along the same orientation.

The first etch mask pattern is preferably masking exactly the area corresponding to the fin of each of the one or more shanks of the probe to be manufactured. This permits the definition of the fins during step (a2(ii)).

Step (a2(ii)) is preferably performed by an anisotropic etching method such as but not limited to reactive ion etching, chemistry enhanced reactive ion etching, magnetron enhanced reactive ion etching, plasma etching and the likes.

In an embodiment, the method according to the first aspect may further comprises a step (s) after step (a1) and before step (a2(ii)), of providing on the second surface, a second etch mask pattern delimiting the shape of the shanks of the probe to be fabricated, the second etch mask pattern being resistant to an etching process. This permits to thin the part of the substrate corresponding to the shanks to be manufactured, thereby defining the shanks.

Preferably, step (s) is performed before step (a2(i)). This permits the first etch mask pattern to be closer to the substrate in the area delimiting the shape of the shanks than the second etch mask pattern.

In an embodiment of the first aspect, the second etch mask pattern may further mask the area corresponding to the connector part of the probe to be manufactured. This permits to prevent the connector part to be thinned when the first etch mask pattern is removed to enable the total thickness (including the fin) of the shanks to be reduced while keeping the height of the fin substantially constant.

In an embodiment of the first aspect, in step (a2(i)), in addition to masking the area corresponding to the fin, the first etch mask pattern may further mask the area corresponding to the connector part of the probe to be manufactured by covering the second etch mask pattern. Preferably, in addition to masking the area corresponding to the fin, the first etch mask pattern covers exactly the second etch mask pattern.

In an embodiment involving a step (s), step (a2(i)) may comprise providing a first etch mask pattern on the second surface, the first etch mask pattern masking exactly the area corresponding to the fin of each of the one or more shanks, masking the area corresponding to the connector part and delimiting the shape of the shank of the probe to be manufactured by (preferably exactly) covering the second etch mask pattern, the first etch mask pattern being resistant to an etching process.

In an embodiment, the first etch mask pattern and the second etch mask pattern have a different chemical nature in order to permit the selective removal of the first etch mask pattern. In an embodiment, the first etch mask pattern can be made of any photoresist (either positive or negative). In an embodiment, the first etch mask pattern can be made of any polymer. In another embodiment, the first etch mask pattern can be made of a metal (e.g. aluminum) that is compatible with the etching process. In an embodiment, the second etch mask pattern is preferably made of silicon oxide deposited using chemical vapor deposition. In another embodiment, it can be made of metal if compatible with the process.

In an embodiment of the first aspect involving a step (s), the method may further comprise after step (a2(ii)), a step of:

a2(iii). removing the first etch mask pattern without removing the second etch mask pattern, and

a2(iv). etching the second surface with the etching process for which the second etch mask pattern is resistant, in a direction perpendicular to the second surface in such a way as thinning but not eliminating at least part of the substrate away from the second etch mask pattern, thereby defining the thickness of the shanks.

a3. at least partially cutting out the probe from the substrate.

In this embodiment, step (a2(iv)) reduces the thickness of the shank without reducing the height of the fin.

In an embodiment, step (s) may comprise providing on the second surface, a layer covering the orthogonal projection on the second surface of the shape of the probe to be fabricated, the layer being resistant to the etching process, and patterning the layer by creating an opening in the layer, the opening comprising the orthogonal projection of the shanks of the probe to be fabricated.

The layer deposited in step (s) may for instance be deposited by chemical vapor deposition.

In an embodiment of the first aspect, a masking layer may be present on the first surface of the substrate below the at least one recording and/or stimulating site and wherein step (a3) is performed by patterning the masking layer so as to form openings in the masking layer around the shape of the probe to be fabricated and subsequently etching the first surface. This has the effect of etching through the first surface of the substrate wherever openings are present. If these openings are defining the complete contour of the probe to be fabricated, this etching sets the probe free from the rest of the substrate.

In a preferred embodiment of the first aspect, the present disclosure relates to a method for manufacturing a probe for brain recording and/or stimulation, the probe comprising a connector part from which one or more shanks protrude along a same orientation, wherein each of the shanks has a first side and a second side opposed to the first side and comprises at least one recording and/or stimulating site on the first side and a fin protruding perpendicularly from the second side and running on at least a part of the length of the shank along the same orientation, thereby preferably providing the shank with a T-shaped cross-section, the method comprising the steps of:

a1) providing a substrate having a first and a second surface opposed to the first surface, the first surface comprising the at least one recording and/or stimulating site of each of the one or more shanks of the probe to be manufactured,

s) providing on the second surface, a second etch mask pattern delimiting the shape of the shanks and masking the area corresponding to the connector part of the probe to be manufactured, the second etch mask pattern being resistant to a etching process,

a2(i)) providing a first etch mask pattern on the second surface, the first etch mask pattern masking exactly the area corresponding to the fin of each of the one or more shanks, masking the area corresponding to the connector part and delimiting the shape of the shank of the probe to be manufactured by (preferably exactly) covering the second etch mask pattern, the first etch mask pattern being resistant to an etching process,

a2(ii)) etching the second surface with an etching process for which the first etch mask pattern is resistant, in a direction perpendicular to the second surface in such a way as thinning, but not eliminating, at least part of the substrate away from the first etch mask pattern, thereby defining the height of the fins,

a2(iii)) removing the first etch mask pattern without removing the second etch mask pattern,

a2(iv)) etching the second surface with an etching process for which the second etch mask pattern is resistant, in a direction perpendicular to the second surface in such a way as thinning but not eliminating at least part of the substrate away from the second etch mask pattern, thereby defining the thickness of the shanks, and

a3) at least partially cutting out the probe from the substrate.

In an embodiment of the present disclosure, the patterning of the masking layer may be such that at least one attachment point between the probe and the substrate outside of the probe is maintained upon performing step (a2(iv)) and whereby the attachment point permits the detachment by hand of the probe from the rest of the substrate. This is the case where the openings are defining only part of the contour of the probe to be fabricated (i.e. portions of the contour are without opening in the masking layer). In this case, the etching only partly frees the probe from the rest of the substrate, i.e. attachment points remain between the probe and the rest of the substrate. These portions of the contour without opening in the masking layer are preferably present around the connecting part of the probe. The connecting part of the probe being less fragile than the shanks, there is less risk of breaking the probe upon manually detaching the probe from its substrate.

In an embodiment of the first aspect, the method may further comprise a step (b) of sharpening the distal end of the shanks. Shanks which are not sharpened at their distal end have difficulties in piercing through the brain membrane (also known as “Dura matter”). Sharpening the distal end of the shanks permits to avoid harmful dimpling of the Dura matter upon implantation of the probe through the Dura matter.

The sharpening is preferably performed by grinding the second side of the shanks. This technique may be performed by temporary fixing the unsharpened probe on a carrier (or jig) in such a way that the distal ends of the shanks are leading over the carrier. The carrier may be placed against a rotating disk and have thereby the second side of the shanks distal ends grinded under the same angle as the carrier.

Shanks sharpen by grinding of their second side are easy to implant through the Dura matter of a rhesus monkey. Since the Dura does not need to be opened, the risk of infection during surgery is lowered.

Other ways to sharpen the distal ends of the shanks are for instance using selective anisotropic etching techniques or wet etching techniques using high dopant implantation into a silicon substrate.

According to an embodiment of the method, a probe is provided, the probe comprising a connector part, and parallel shanks extending outward from the connector part, the shanks having an essentially constant thickness extending between a first and second side of the shank, and the fin protruding perpendicularly from the second side of the shank being attached to the second surface of the carrier, with the connector part and the majority of each shaft portion facing the second surface (e.g. attached via the fin or via other means), and the distal end (e.g. a triangular chisel shaped tip portions) being in close proximity to the edge, and wherein material is removed from the distal end of all of the shanks.

According to an embodiment, at least part of the distal end(s) is (are) extending outward from the edge, so that the part of the distal end(s) extend(s) below the first surface of the carrier when the first surface is held horizontally, and wherein substantially no material of the carrier is removed during the grinding step.

According to another embodiment, the shank(s) may not extend outward from the edge, and wherein the carrier material and the shank tip material are removed simultaneously during the grinding step.

The edge may be shaped as a straight line. The shank(s) may be placed perpendicularly to the edge.

The location on the carrier block onto which the shank(s) is (are) attached may be provided with an alignment structure. The alignment structure may comprise one or more grooves into which the shank or shanks may be placed. The alignment structure may be a negative imprint of the probe. In an embodiment, the alignment structure may comprise on or more groves in which the fins may be placed.

According to an embodiment, the grinding step is done by placing the carrier on a rotating grinding disc and applying pressure on the carrier. The grinding step may be followed by a polishing step.

According to an embodiment, the grinding step is continued so as to obtain a sharp pointed tip, lying in the plane defined by the first side of the shank or probe.

According to another embodiment, the grinding step is followed by the steps of:

attaching the first side of the probe to the second surface of the carrier, with the connector part and the majority of the shaft portion of each shank attached to the second surface and the distal end (tip portion) being in close proximity to the edge,

subjecting the first surface of the carrier and at least the distal end (e.g. part of the tip portion) of the shank(s) to a grinding step, to thereby remove material at least from the distal end (e.g. a triangular, chisel shaped tip portion) of the probe(s), so as to obtain a sharp pointed tip lying in between the planes defined by the first and second side of the shank or probe.

In a second aspect, the present disclosure relates to a probe for brain recording and/or stimulation, the probe comprising one or more shanks having a same orientation, wherein each of the shanks has a first side and a second side opposed to the first side and comprises at least one recording and/or stimulating site on the first side and a fin protruding perpendicularly from the second side and running on at least a part of the length of the shank along the same orientation. Each shank comprises an elongate shaft portion and a tip portion. The tip portion is at the distal end of the shanks. The tip portion may have a triangular chisel-shape. The shafts have an essentially constant thickness extending between the first side and the second side.

In an embodiment of the second aspect, the shanks may have a T-shaped cross-section provided by the fin protruding perpendicularly from the second side.

In other words, the second aspect of the present disclosure may relate to a probe for brain recording or stimulation, the probe comprising shanks having a T-shaped profile, the T-shape being defined by a horizontal part and a vertical part, wherein the horizontal part has a first side and a second side, wherein the vertical part is attached to the second side, and wherein the first side comprises one or more recording and/or stimulating sites (e.g. electrodes).

In an embodiment of the second aspect, the probe may further comprise a connector part from which the one or more shanks protrude along the same orientation.

In an embodiment, there may be a shorter distance between the first and the second side of the shanks at the distal end of the shanks than away from the distal end. This sharpens the distal end of the shanks and helps to pierce through the Dura matter with a reduced dimpling effect.

In an embodiment, the first and the second surface of the shanks are not parallel at the distal end of the shanks. This sharpens the distal end of the shanks.

In an embodiment, the first and the second surface of the shanks may intersect in a line at the distal end of the shanks, the line producing an edge. This edge is thereby made sharp which permits it to cut through the Dura matter without dimpling effect.

In an embodiment, the first side may have a same orientation at the distal end of the shank and away from the distal end while the second side at the distal end makes an angle with the second side away from the distal end. In other words, a sharp pointed tip may be present at the distal end of the shank, the pointed tip lying in the plane defined by the first side of the shank or probe.

In another embodiment, the present disclosure may relate to a probe for brain recording and/or stimulation, the probe comprising one or more shanks having a same orientation, wherein each of the shanks has a first side and a second side opposed to the first side and comprises at least one recording and/or stimulating site on the first side and a fin protruding perpendicularly from the second side and running on at least a part of the length of the shank along the same orientation, each of the shanks having a sharp pointed tip at its distal end, the tip lying in the plane defined by the first side.

This is the geometry obtained by grinding the second side of the shafts. This permits to avoid damages to the first side of the shafts (which comprise recording/stimulating sites) while sharpening the shafts. Such a sharpening would typically deviate the shanks from their trajectory upon insertion by inducing a bending upward (in the direction of the first side) of the shanks. However, this bending is efficiently prevented by the fin protruding perpendicularly from the second side of the shanks of the present disclosure. There is therefore synergy between the presence of the fin and the presence of the sharpening. An alternative way to prevent this bending would be to manufacture probes having shanks with a sharp pointed tip lying in between the planes defined by the first and second surface of the shank or probe. This would prevent bending in so far as the tip would be lying exactly at mid-distance between the planes defined by the first and second surface of the shank or probe. The manufacture of such tips is however delicate and requires two grinding steps while embodiments of the present disclosure permit to prevent bending even of tips obtained by only one grinding step.

In an embodiment, the shanks may have a triangular and/or chisel shaped tip at their distal end.

In an embodiment, each of the shanks may have a total length of 0.5 to 10 mm.

In an embodiment, the fin may have a rectangular parallelepiped shape.

In an embodiment, the shank may have a thickness (fin included) of from 15 to 100 μm, preferably from 15 to 50 μm, even more preferably from 15 to 30 μm and most preferably from 20 to 30 μm. In other words, this corresponds to the total height of the T-shape. This total height is preferably higher than 15 μm and more preferably higher than 20 μm in order to assure enough rigidity to the shanks. Below 15 μm, the bending tends to be too high.

In an embodiment, the shanks may have a thickness away from the fin (and from their distal end if the shanks are sharpened by reducing their thickness at their distal end) of from 1 to 20 μm, preferably 1 to 15 μm. Typically, when this thickness is 20 μm or lower, the rigidity of the shank (without fin) might be too low to assure proper penetration in the brain. In such a case, the presence of the fin is particularly advantageous.

In an embodiment, the fin may have a height of from 1 to 35 μm.

In an embodiment, the fin may have a width of from 1 to 30 μm, preferably from 5 to 20 μm.

In an embodiment, the width of the shanks away from their distal end may be from 40 μm to 200 μm, preferably from 40 μm to 140 μm, more preferably from 40 μm to 120 μm and even more preferably from 40 μm to 100 μm. The width of the shanks preferably is reduced (preferably to a point) at their distal end. This eases the penetration of the shanks in the brain.

In an embodiment, the width of the fin maybe from 2 to 30 μm, preferably from 5 to 20 μm.

The area of a shank's cross-section (e.g. T-shaped cross-section) is preferably smaller than 6000 μm², more preferably smaller than 2500 μm² and still more preferably smaller than 1250 μm² in order to minimize harm to the tissues. For instance this area can be from 500 to 1250 μm².

In an embodiment, the ratio between (i) the width of the shank away from its distal end and (ii) the thickness (fin included) of the shank can be from 1 to 3.5. Above 3.5, the bending of the shank tends to be too high.

In an embodiment, the probe may comprise at least one shank being a first shank according to any embodiment of the second aspect, which fin is further attached to either:

a) the fin of a second shank according to any embodiment of the first aspect, or

b) the second side of a second shank having a first side and a second side opposed to the first side and comprising at least one recording and/or stimulating site on the first side, or

c) the second side of a second shank having a first side and a second side opposed to the first side, thereby providing an H-shaped profile to the shank.

In an embodiment, the fin of the second shank may belong to a second probe for brain recording and/or stimulation, comprising one or more shanks having the same orientation.

Such an H-shaped profile further rigidifies the shank and provides in cases (a) and (b) two surfaces for recording and/or stimulating. Such an H-shaped shank is easier to insert in a brain than a flat shank of total thickness lower than the total thickness of the H-shaped profile. The reason is that in the H-shaped profile, each of the two shanks composing the H-shape can be thinner than the flat shank, even if the total structure is thicker. The fin being relatively thin, it does not interfere much with the penetration of the H-shaped shank.

In a third aspect, the present disclosure relates to a method of fabricating a probe for brain recording or stimulation, the probe comprising a shank having a H-shaped cross-section, the method comprising the step of attaching the free extremity of the fin of a shank of a probe according to any embodiment of the second aspect to either:

a) the fin of a second shank according to any embodiment of the second aspect, or

b) the second side of a second shank having a first side and a second side opposed to the first side and comprising at least one recording and/or stimulating site on the first side, or

c) the second side of a second shank having a first side and a second side opposed to the first side.

In an embodiment, the fin of the second shank may belong to a second probe for brain recording and/or stimulation, comprising one or more shanks having the same orientation.

In a fourth aspect, the present disclosure relates to a probe comprising one or more shanks having a same orientation, wherein each of the shanks has a first side and a second side opposed to the first side and comprises at least one recording and/or stimulating site on the first side which second side is attached to the second side of a second shank having a first side and a second side opposed to the first side and comprising at least one recording and/or stimulating site on the first side, thereby providing a probe having two parallel sets of shanks, wherein each shank in the first set is linked to a shank in the second set. In an embodiment, the distance between two adjacent shanks in the first set is equal to the distance between two adjacent shanks in the second set.

In other words, in the fourth aspect, the probe comprises one or more composite shanks, each of the composite shanks being formed of two component shanks attached back to back (second side to second side).

Such a probe has the advantage to have recording and/or stimulating sites on both its sides. Such a probe has the further advantage that the two component shanks have a tendency to bend in opposite directions of one another (due to the internal stress of the various thin film layers used in the probe fabrication), attaching them back to back cancels this tendency and gives rise to a surprisingly rigid probe. This is especially advantageous when the probe has integrated CMOS.

Such a probe has the further advantage that it can be made relatively thin. Each component shank may have a thickness of from 8 to 50 μm, preferably 8 to 25 μm and most preferably 8 to 15 μm. Therefore, the composite shanks may have a thickness of from 16 to 100 μm, preferably 16 to 50 μm and most preferably 16 to 30 μm. The length and the width away from the distal end can be as described for the second aspect of the present disclosure. An intermediate layer of a dissimilar material (for instance a metal or a polymer) can also be attached in between the two component shanks to modify the probe resistance to bending and/or breakage.

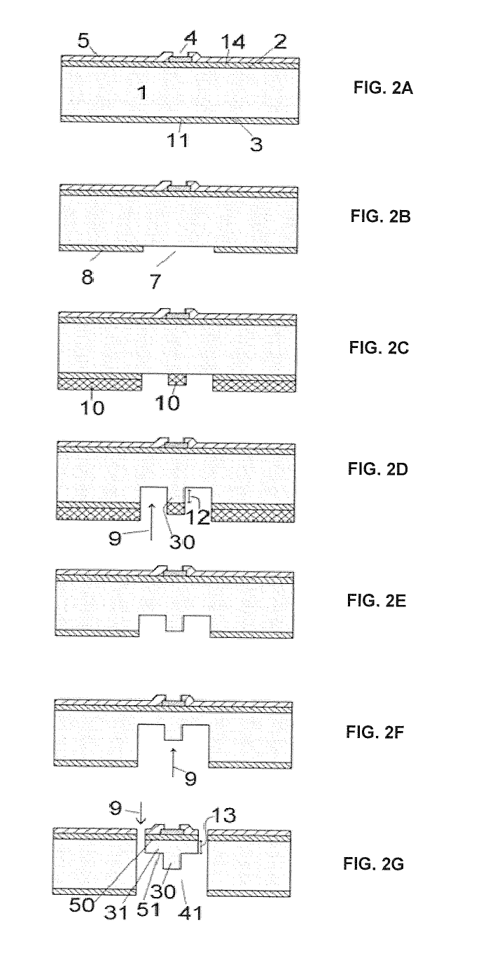

In an embodiment of the first aspect, the present disclosure relates to a method of fabricating a probe for brain recording or stimulation comprising shanks having a T-shaped cross-section defined by a vertical part 30 and a horizontal part 31 (illustrated orthogonal to the probe through one electrode in FIG. 2). As shown in FIG. 2A, the method comprises providing a substrate 1 having a first 2 and a second 3 surface, whereby the first surface 2 comprises at least one electrode 4. In a following step, as shown in FIG. 2C, a first etch mask pattern 10 is provided on the second surface 3. Optionally, an isolation layer 14 is positioned directly under the electrode 4. The surface created by orthogonal projection of the electrode onto the second surface overlaps the first etch mask pattern 10. As shown in FIG. 2D, dry-etching of the second surface 3 is performed in a direction 9 perpendicular to the second surface 3 in such a way as thinning, but not eliminating, at least part of the substrate 1 away from the first etch mask pattern 10, thereby defining the height 12 of the vertical part 30 in the T-shaped cross-section.

In another embodiment, as shown in FIG. 2B, the method may further comprise providing a second etch mask pattern 8 on the second surface 3, surrounding but not overlapping the area corresponding to the area of the orthogonal projection of the electrode 4, and whereby the second etch mask pattern 8 is resistant to a dry etching process. Preferably a method step illustrates by FIG. 2B is performed before a method step illustrated by FIG. 2C.

Preferably after the electrodes are defined in the first surface 2, according to an embodiment of the disclosure, passivation can be applied on the first surfaces of the substrate. The substrate material used is preferably a semiconductor and more specifically silicon.

The electrode can be deposited using several deposition techniques known in the art, such as for example electron beam evaporation, sputter deposition or plasma vapor deposition or any other conventional thin film deposition technique.

In yet another embodiment, as shown in FIG. 2A, a layer 11 may be provided on the second surface 3, the layer 11 covering the second surface 3 and whereby the layer 11 is resistant to a dry etching process. By patterning the layer 11 in such a way as to create openings in the layer, whereby an opening preferably comprises the area resulting from the orthogonal projection of the electrode 4 on the second surface 3, a first etch mask pattern 8 is fabricated.

Preferably the first and second etch mask patterns can comprise any material capable of blocking the dry-etching, in selected areas of the substrate; typically photoresist can be used as an etch mask pattern. Photoresist can be any photo-sensitive material used in photolithography that can transfer a pattern from the mask onto the substrate.

Several dry-etching techniques can be applied for fabricating the probe according to any embodiments of the disclosure, for instance Xenon difluoride etching, reactive ion etching or deep reactive ion etching. Preferably deep reactive ion etching is used to etch away parts of the substrate 1. Advantageously, by using this etching process, etch depths of hundreds of micrometers are achieved with almost vertical sidewalls. The primary technology is based on the so-called patented “Bosch process” (FR 2948495). The process can easily be used to etch completely through a silicon substrate, and etch rates are 3-6 times higher than wet etching.

In some other embodiments, as shown in FIGS. 2D and 2E, the method may further comprise removing the first etch mask pattern 10 without removing the second etch mask pattern 8, and then, as shown in FIG. 2F, dry-etching of the second surface 3 is performed perpendicularly (see arrow 9 in FIGS. 2D-2F) to the second surface 3. As a result, the substrate is thinned but not eliminated, thereby defining the thickness 13 of the horizontal part 31 in the T-shaped cross-section 32. As shown in FIG. 2G, the portion of the substrate lying within the second mask 8 is at least partially cut out from the substrate 1, thereby providing the probe (if entirely cut out) or provide the probe still attached via attachment points to the rest of the substrate 1 (if partially cut out).

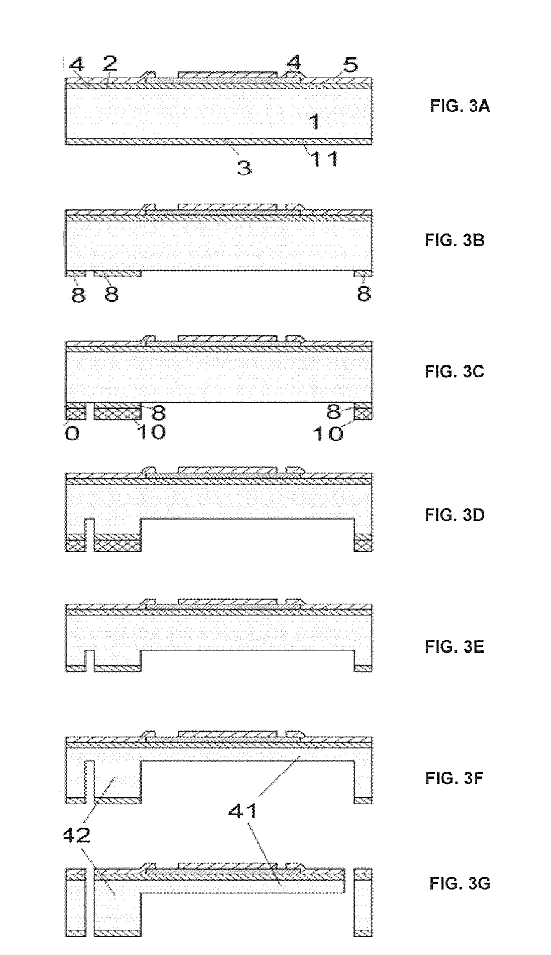

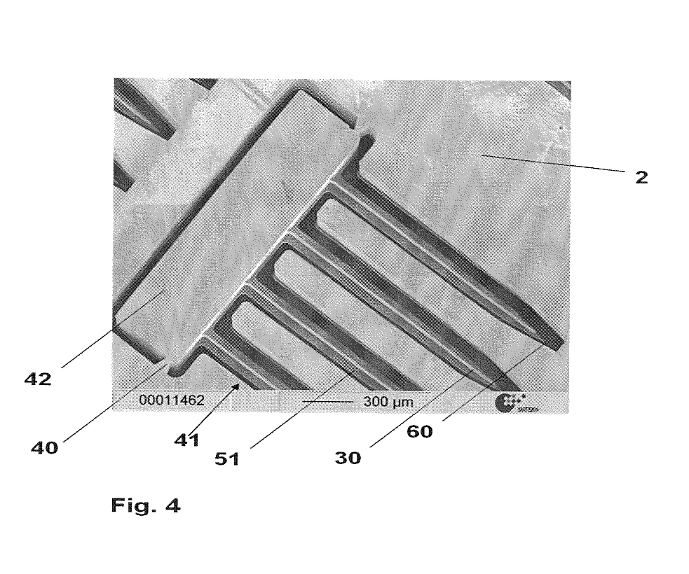

Preferably, the partial cutting of the probe can be achieved by dry-etching the first surface 2 of the substrate 1 after patterning of an isolation layer 14 and passivation layer 5 on the first surface 2 of the substrate 1. In FIG. 3F, the partial cutting is illustrated in a clear way where the connector part 42 is attached to the substrate 1 via an attachment point 40. The step associated with FIG. 3G is performed either everywhere or not everywhere (via appropriate masking of the first surface) in order to maintain attachment points 40. In FIG. 4 the partial cutting of the probe is illustrated as seen from the second surface of the substrate. Preferably at least one attachment point 40 between the connector part 42 and the substrate 1 remains after the partial etching and thus cutting of the first surface. Preferably the attachment point 40 is easily released from the rest of the substrate. For instance, using tweezers to release the probe from the substrate can be easily performed. Advantageously as a result, no additional dicing is required to separate the probes from the fabrication substrate or wafer.

An advantage of a method in accordance with embodiments of the present disclosure is that it offers a solution for drastically reducing the cross-sectional area of a probe without compromising the resistance of the probe to bending, buckling or flexing. As a result of this drastically reduced cross-section the amount of damage the probe inflicts to the biological tissue is as low as possible. However, when making the probes too thin other problems such as increased fragility, flexing or buckling can occur during the insertion phase of the probe. Advantageously, the T-shaped cross-section additionally provides reinforcement of the probe in order to overcome these problems.

A probe 32 for brain recording or stimulation, according to one embodiment of the disclosure, comprises a T-shaped profile and the T-shape is defined by a horizontal part 31 and a vertical part 30, wherein the horizontal part has a first surface 50 and a second surface 51, wherein the vertical part 30 is attached to the second surface 51, and wherein the first surface 50 comprises one or more electrodes (recording/stimulating sites) 4.

Preferably the probe according to any embodiments has a sharp distal end and has a stiffness that is sufficient for allowing insertion of the probe into a brain. More specifically the probe can be inserted in the brain without deflection.

Another embodiment of the present disclosure relates to a method of fabricating a probe for brain recording or stimulation, whereby the probe has shanks having a H-shaped cross-section defined by a vertical part and two horizontal parts. The method comprises the steps of attaching the free extremity of the vertical part of a shank according to anyone of the previous embodiments with either a further horizontal part or the free extremity of a vertical part of a second shaft according to anyone of the previous embodiments.

An advantage of using a probe with H-shaped cross-section shanks is that it doubles the amount of electrodes that can be used: the electrodes on the first surface of the first and second horizontal parts of the H-shaped cross-sectioned shanks.

Preferable the attaching of the free extremities of the vertical part of both shanks can be enabled by glue or anodic bonding. Any glue suitable for attaching electronic components can be used. Before applying the glue the free extremities are preferably cleaned.

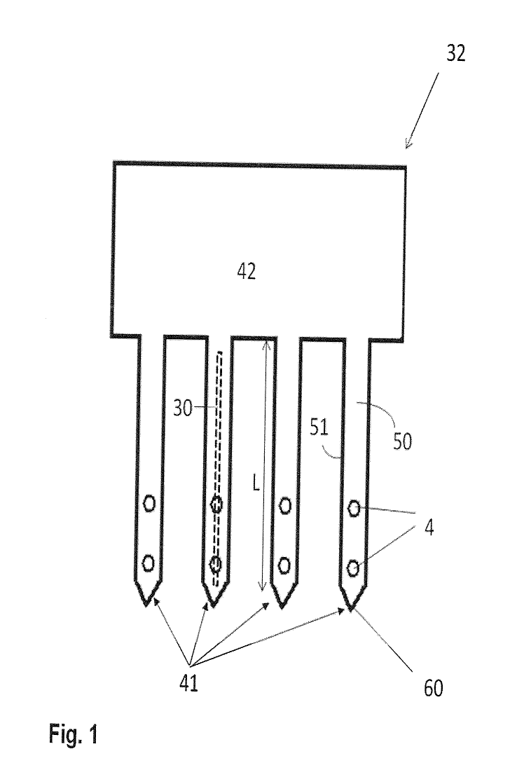

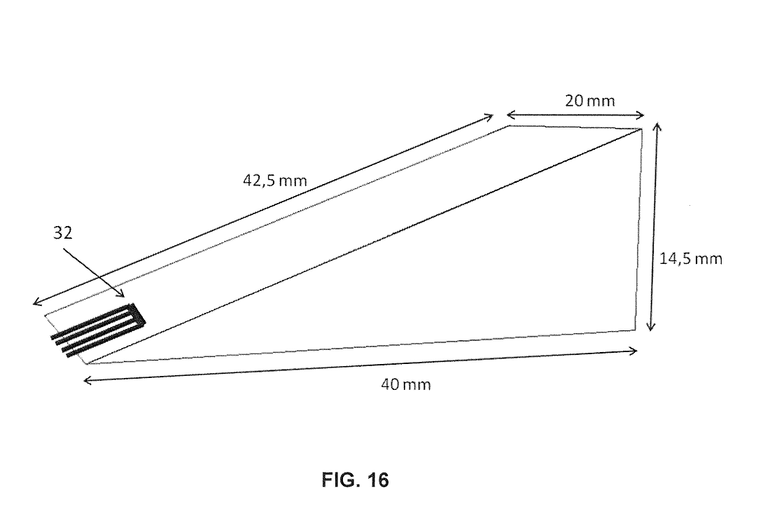

Each of FIGS. 1-16 is hereafter described in more detail.

FIG. 1 schematically illustrates a probe 32 for brain recording and/or stimulation according to an embodiment of the present disclosure. It comprises four shanks 41 and a connector part 42. From the connector part 42, each shank 41 protrudes along a same orientation. The shank 41 comprises a first side 50 and a second side 51 opposite to the first side. The first side 50 of the shank 41 comprises a number of recording and/or stimulating sites 4 that are connected to a signal processing circuitry. The second side 51 of the shank comprises a fin 30, protruding perpendicularly from the second side 51 and running on a major part of the shank 41 along the same orientation as the shank. The thickness of the elongate shaft portions and the triangular chisel-shaped tip portion is essentially constant. The probe has a first and second flat surface, the thickness of the probes extending between the surfaces. When the probe 32 is inserted in the brain, the shanks 41 penetrate the brain tissue. The penetrating of the shanks 41 is aided by a sharpened distal end 60 of the shank. This distal end 60 of the shank 41 is the furthermost point of the shank in relation to the point of attachment with the connector part 42.

FIGS. 2A-2G illustrate a schematic representation of a manufacturing method viewed orthogonally to the probe shank 41 through one recording and/or stimulating site 4, according to an embodiment of the present disclosure. As shown in FIG. 2A, the method comprises a step of providing a substrate 1 having a first 2 and a second 3 surface, whereby the first surface 2 comprises at least one recording and/or stimulating site 4. As shown in FIG. 2C, a first etch mask pattern 10 is provided on the second surface 3, and the first etch mask pattern 10 masks the area corresponding to the fin 30 of the shank 41. As shown in FIG. 2D, a dry-etching of the second surface 3 is performed in a direction 9 perpendicular to the second surface 3 in such a way as thinning, but not eliminating, at least part of the substrate 1 away from the first etch mask pattern 10, thereby defining the height 12 of the fin 30 of the shank 41. The first etch mask pattern 10 is resistant to the dry-etching process performed, as shown in FIG. 2E.

Preferably after the recording and/or stimulating sites 4 are defined a passivation layer 5 is provided before starting the manufacturing process. Preferably, an etch mask layer 11 and an isolation layer 14 on respectively the second surface 3 and the first surface 2 of the substrate 1 can be applied. The substrate material used is preferably a semiconductor and more specifically silicon. The recording and/or stimulating sites 4 can be deposited using several deposition techniques known in the prior art, such as for example electron beam deposition, sputtering or plasma vapor deposition or any other conventional thin film deposition technique.

In another embodiment, as shown in FIG. 2B, the method may further comprise a step of providing a second etch mask pattern 8 on the second surface 3, delimiting the shape of the shanks 41 of the probe 32 to be fabricated. The second etch mask pattern 8 can be created by patterning the etch mask layer 11 covering the second surface 3 in such a way as to create an opening 7 in the layer, whereby the opening 7 preferably comprises the masking area corresponding to the fin 30 of the probe 32. Preferably, the method step associated with FIG. 2B is performed before the method step associated with FIG. 2C. Preferably at least part of the masking area 8 corresponds to the connector part 42 of the probe 32. The second etch mask pattern 8 is resistant to a dry-etching process.

Several dry-etching techniques can be applied for fabricating the probe according to any embodiments of the disclosure, for instance Xenon difluoride etching, reactive ion etching or deep reactive ion etching. Preferably deep reactive ion etching is used to etch away parts of the substrate 1. Advantageously, by using this etching process, etch depths of hundreds of micrometers are achieved with almost vertical sidewalls. The primary technology is based on the so-called patented “Bosch process” (FR 2948495). The process can easily be used to etch completely through a silicon substrate, and etch rates are 3-6 times higher than wet etching.

In some other embodiments, as shown in FIG. 2E, the method further can comprise a step of removing the first etch mask pattern 10 without removing the second etch mask pattern 8, and then, as shown in FIG. 2F, a step of dry-etching of the second surface 3 is performed in a direction perpendicular 9 to the second surface 3. As a result of this FIG. 2F step, the substrate is thinned but not eliminated, thereby defining the thickness 13 of the horizontal part 31 of the shank 41 having T-shaped cross-section, and thus also defining the thickness of the shank 41. As shown in FIG. 2G, in a final step the probe 32 is partially cut out from the substrate 1, thereby providing the probe 32. Preferably, the partial cutting of the probe 32 can be achieved by dry-etching the first surface 2 of the substrate 1 after patterning of an isolation layer 14 and the passivation layer 5 on the first surface 2 of the substrate 1.

Referring to FIGS. 3A-3G, the partial cutting is illustrated in a clear way in FIG. 3F and FIG. 3G. In FIG. 3F, the connector part 42 is attached to the substrate and the shanks 41 are fabricated. As shown in FIG. 3G, the cutting is performed and looking along the probe shank 41 the probe is completely cut out.

Moreover, in FIG. 4 the partial cutting of the probe is illustrated as seen from the second surface 3 of the substrate 1. Preferably at least one attachment point 40 between the connector part 42 and the substrate 1 remains after the partial etching and thus cutting of the first surface. Preferably the attachment point 40 is easily released from its coupled state. For instance, using tweezers to release the probe from the substrate can be easily performed.

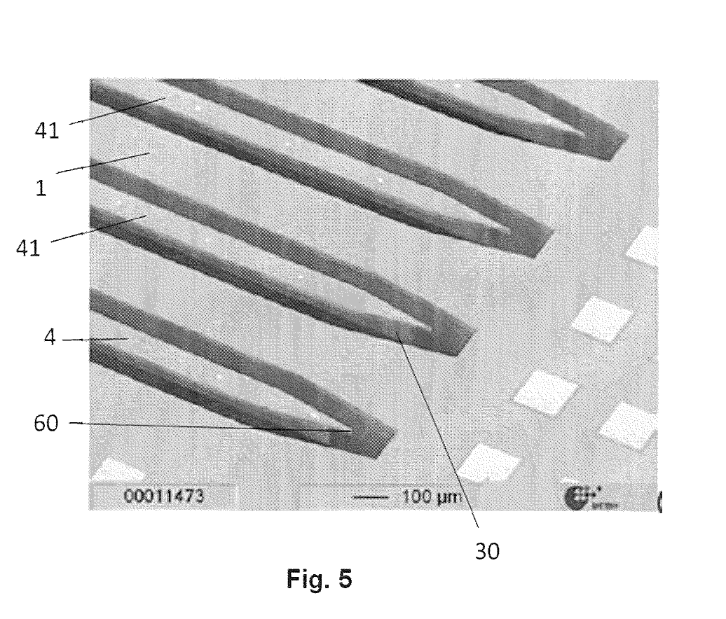

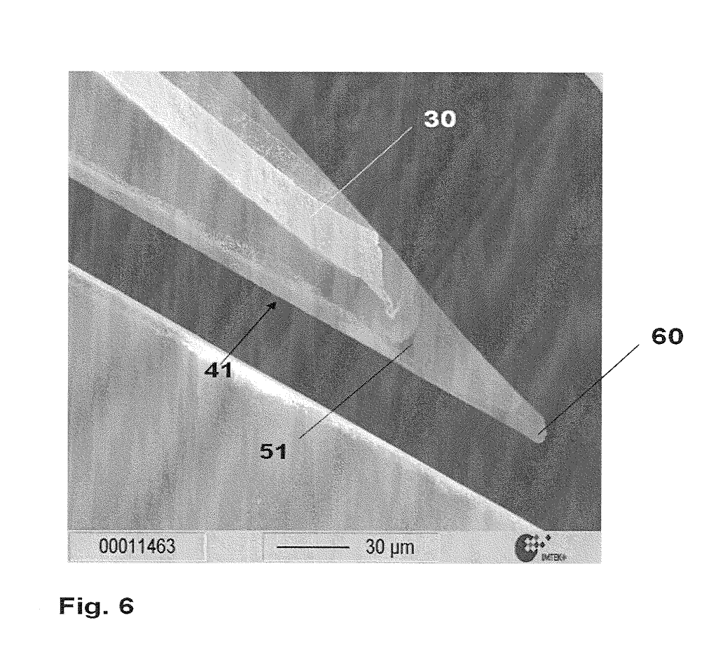

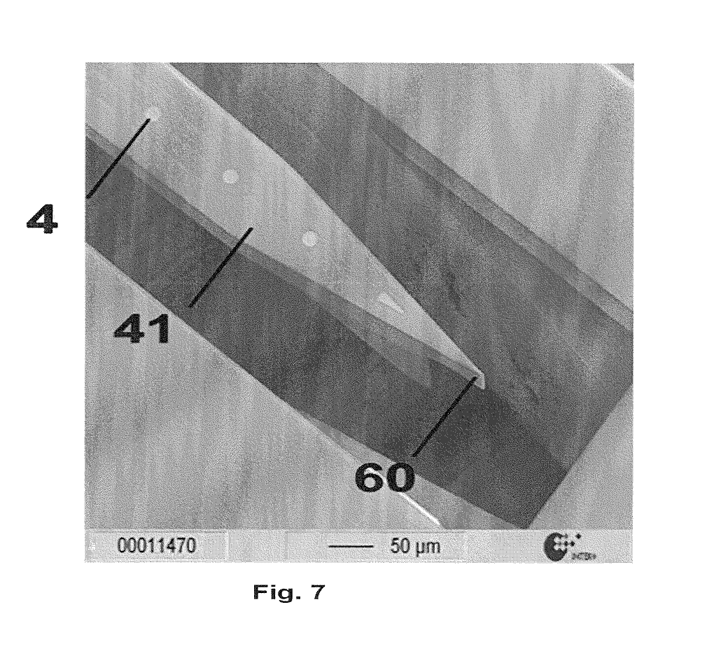

FIG. 5 illustrates the shanks 41 have a T-shaped cross-section, whereby the fin 30 defines the vertical part of the T-shaped cross-section. The distal end 60 of the shanks 41 are preferably sharpen in an additional method step according to some embodiments of the disclosure. FIG. 6 shows an enlarged picture of the fin 30 of a shank 41 seen on second side 51 of the shank. FIG. 7 shows an enlarged picture of the distal end 60 of a shank 41 comprising recording and/or stimulating sites 4.

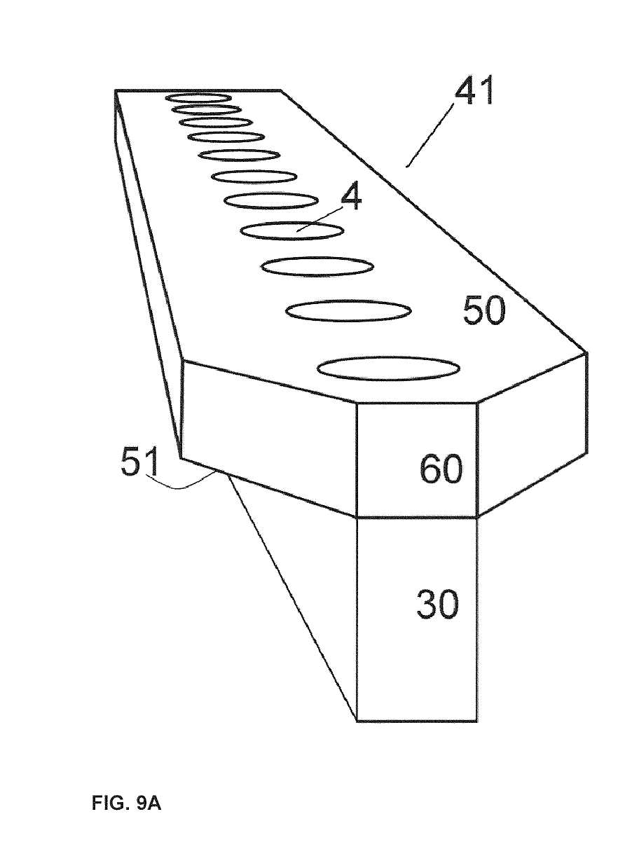

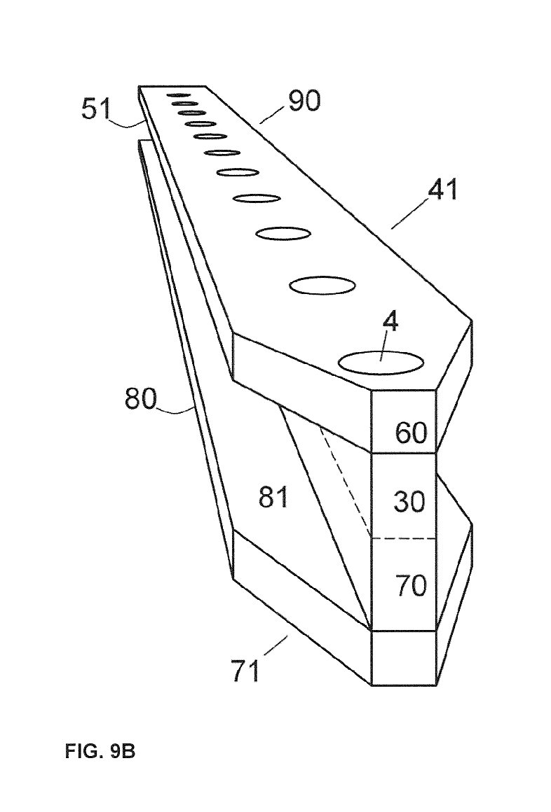

FIG. 9A illustrates a schematic representation of a T-shaped probe shank 41 and FIG. 9B illustrates a schematic representation of an H-shaped probe shank 90. The H-shaped probe shank 90 is constructed by attaching the free extremity of the fin 30 of a shank 41 of a first probe to the fin 70 of a shank 71 of a second probe having a first side 80 and second side 81 that is opposed to the first side. The first side 80 of the second shank 71 comprises at least one recording and/or stimulating sites (not shown). In other embodiments of the disclosure, an H-shaped probe shank 90 can be constructed by attaching the free extremity of the fin 30 of a shank 41 directly to the second side 81 of a second shank 71 comprising no fin 70, whereby the first side of this second shank 71 may comprise recording and/or stimulating sites 4.

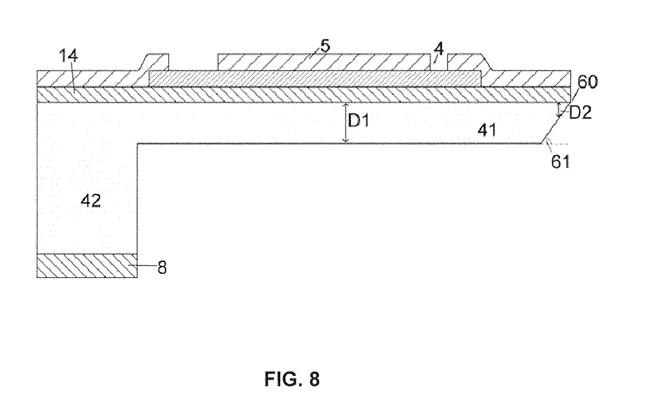

Preferably the sharpening of the distal end 60 can be performed by grinding the second side 51 of the shank. As a result of this sharpening, the height of the horizontal part 13 of the T-shaped cross-section is not uniform throughout the shank length L. This is schematically illustrated in FIG. 8. The thickness D2 of the horizontal part of the T-shaped cross-section 31 at the distal end 60 is smaller than the thickness D1 of the horizontal part of the T-shaped cross-section at a point on the shank 41 away from the distal end 60. More specifically, the second side 51 of the shank 41 comprising the distal end 60 makes an angle 61 with the second side of the shank 41 away from the distal end 60. This angle is smaller than 90° and is preferably smaller than 65°. The tip present at the distal end 60 lies in the plane defined by the probe's first side.

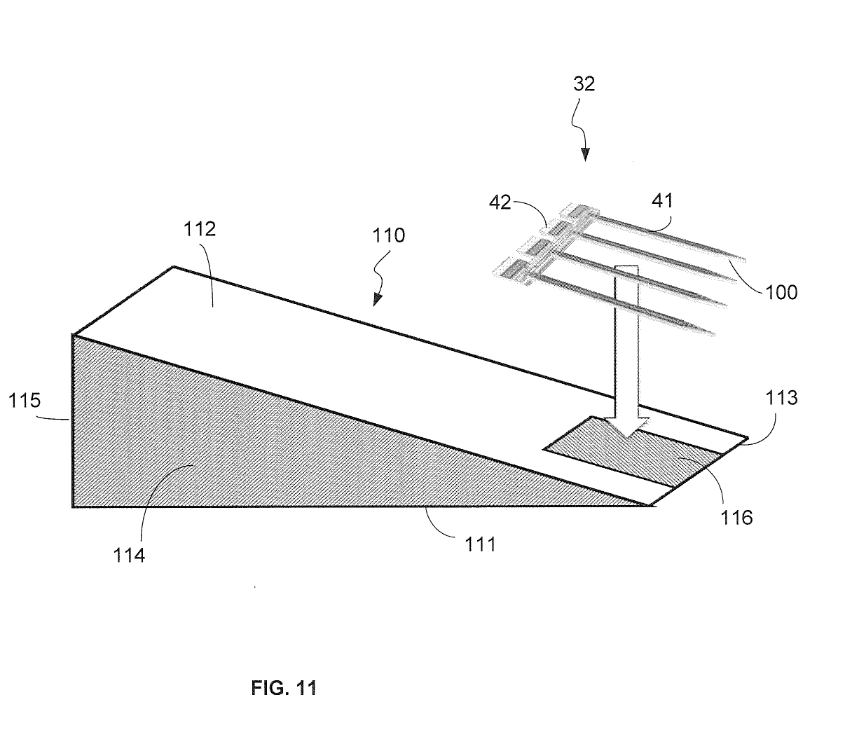

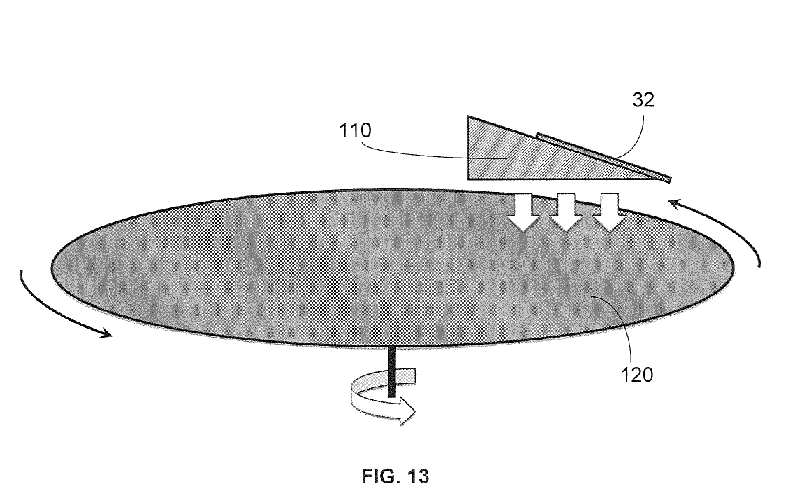

The grinding of the distal end 60 can be performed by temporary fixing the unsharpened probe 32 on a carrier 110 (see FIG. 11), which is sloped under an angle, in such a way that the second side 51 of the shank 41 comprising the distal end of the probe 32 is leading over the carrier 110. The carrier 110 then can be positioned on a rotating polishing disk 120 (see FIG. 13) which grinds the second side 51 of the shank 41 comprising the distal end 60. The carrier 110 with probe 32 can be positioned on a polishing table e.g. for several minutes resulting in a sharp pointy tip. In this example, the above mentioned angle 61 is determined by the angle of the sloped carrier 110 used for grinding. Other methods can also be used for the sharpening of the distal end, for instance by using anisotropic etching techniques that can etch the silicon planes of the probe tip. Also selective etching techniques can be used which apply a high dopant implantation onto the silicon.

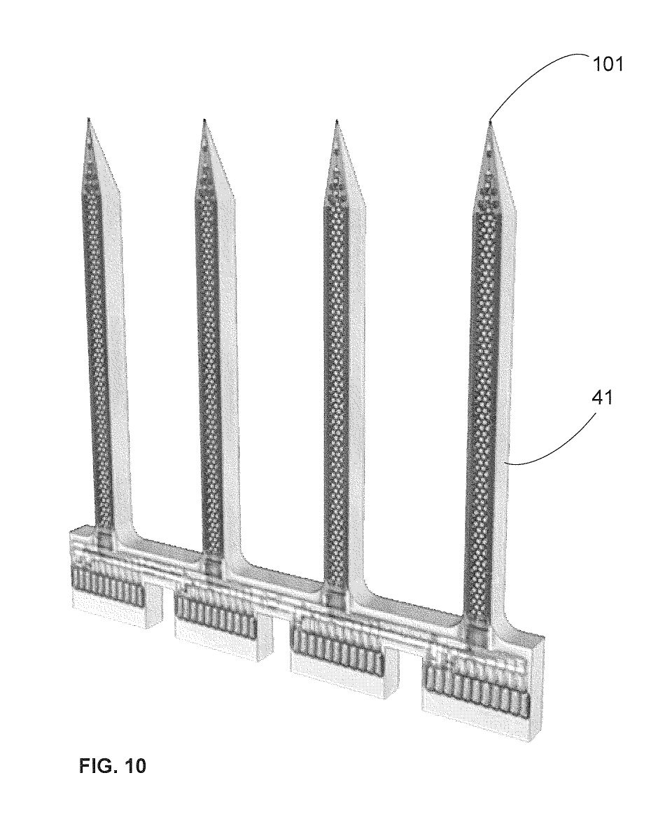

FIG. 10 shows a neural probe obtainable by the method of the disclosure. Each shank has a sharp pointed tip 101, able to pierce through the Dura without the dimpling effect. The fin is not represented but is present.

Preferred embodiments of the method of producing such a probe are illustrated in FIGS. 11-15. The method as described hereafter is also applicable to a single shaft probe. Referring first to FIG. 11, a probe 32 is provided having an essentially constant thickness of the elongate shank 41 and the triangular chisel-shaped tip portions at the distal end, i.e. the probe has a first and second flat side 50/51, the thickness of the shanks extending between the sides. The probe is temporarily attached (e.g. by its fin(s)) at the second side 51, to a carrier 110, which is preferably a solid carrier block, e.g. made of aluminum. The carrier block shown in FIG. 11 is wedge-shaped, with a flat base surface 111 and a slanted top surface 112, forming a sharp angle with respect to the base surface, so that a straight edge 113 is formed between the base and top surfaces 111 and 112. The carrier 110 further comprises parallel side walls 114 and a back wall 115 perpendicular to the base surface. Other outer shapes of the carrier are possible, as long as the carrier comprises a first and second surface 111/112 forming a wedge, i.e. being oriented at a sharp angle with respect to each other, and joined by an edge 113 of the carrier.

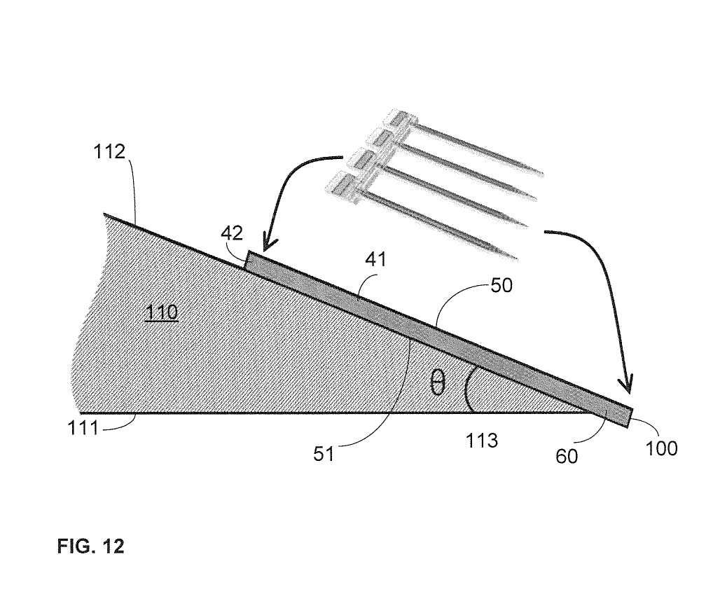

As seen in FIGS. 11 and 12, the probe 32 is releasably attached to a contact location 116 of the carrier's slanted top surface 112, with at least part of the triangular tip portions at the distal end 60 extending over the edge 113, all triangular tip portions being placed at the same distance from the edge 113. This means that a portion of the shanks extends downward from the base surface 111 when the base surface is placed horizontally (see FIG. 12). In the case shown in FIG. 12, the connector part 42 and shanks 41 of the probe 32 are removably attached to the top surface 112 of the carrier block 110 with a suitable temporary adhesive, e.g. a resin or a wax. The contact location 116 on the slanted surface 112 of the block may be provided with an alignment structure, preferably comprising a set of grooves oriented perpendicularly to the edge 113, and at a mutual distance corresponding to the mutual distance of the probes in the array, so that the probes fit into the grooves. More preferably, the alignment structure is a negative imprint of the entire probe array, including the connector part 42, so that the entire probe 32 can be fitted into the carrier block's top surface 112. The above named adhesive is preferably used in addition to the alignment structure. Extending outward from the carrier edge 113 is the straight edge 100 of the chisel-shaped triangular probe tips at the distal end 60. The straight edge 100 is essentially perpendicular to the surface 112 of the carrier.

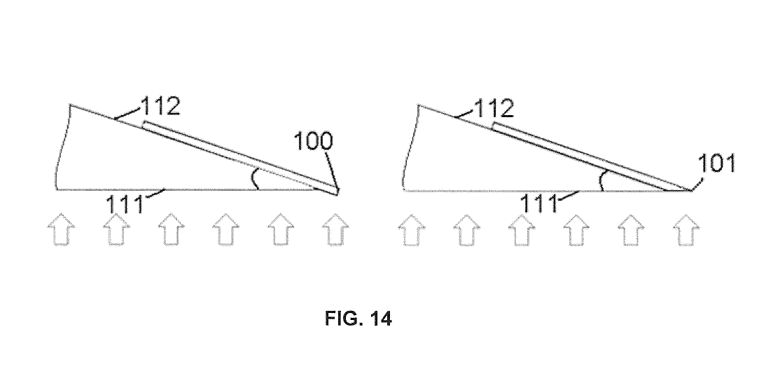

Then the carrier block/probe combination is subjected to a grinding step as illustrated in FIG. 13. Preferably a known grinding/polishing tool is used, having a rotating grinding disc 120 onto which the carrier block 110 can be placed with a suitable pressure. The carrier block can be fixed onto a micro-drive which lowers the carrier onto the rotating grinding/polishing disk. Suitable grinding/polishing tools that can be used are the Saphir 320/330 series from ATM. In the case shown in FIG. 12, the carrier material, the material of the grinding surface, the applied pressure and grinding time are chosen so that substantially no or very little material is removed from the carrier block's base surface 111 during the grinding step, while all the material of the probe tips extending below the base surface 111 is removed during the step. The result of the grinding step is shown in FIG. 14. After a suitable grinding time, the portion of the probes extending below the base surface 111 is removed, and a sharp tip 101 is formed at the distal end of the probes. The angle θ of the carrier block 110 is thereby substantially transferred to the probe tips.

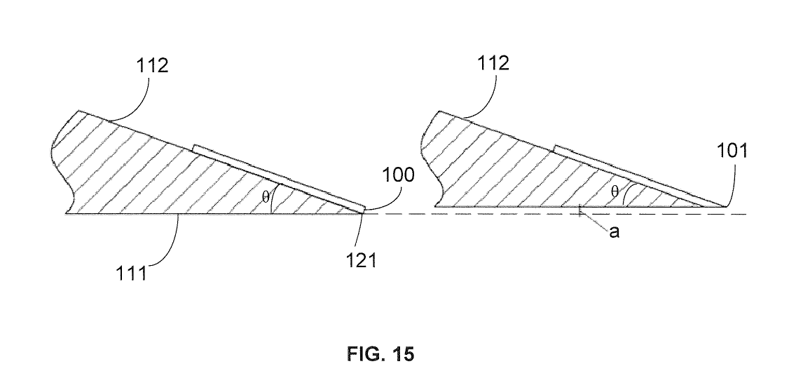

Another embodiment is illustrated in FIG. 15. In this case, the material of the carrier 110, the material of the grinding surface, the grinding pressure and time are chosen so that the carrier block's base surface 111 is thinned during the grinding step, together with the shank tip. The shanks are attached to the carrier block without the triangular tip portions at the distal end 60 extending over the edge 113, preferably with the lower end 121 of the tip placed on the edge 113, as shown in FIG. 15. During the grinding step, a thickness ‘a’ of the carrier block and of the tip material is simultaneously removed, leading also to a sharp tip 101 at the end of the grinding step, with the angle θ of the carrier block transferred to the shank tip. In both embodiments shown in FIGS. 14 and 15, the tip 101 lies in the plane defined by the probe's first side 50.

Variants of the above-described embodiments are included in the scope of the appended claims. The edge 113 may be curved instead of straight. This allows the formation of shank tips of different shape and size in one grinding step. Likewise, instead of placing the shanks of a probe at the same distance to a straight edge 113, it is possible to place the probe at an angle to the edge. Instead of an array of shanks, a single shank may be sharpened by the method of the disclosure. The angle θ is a sharp angle, i.e. lower than 90°, but preferably lower than 45°.

The carrier 110 is preferably but not necessarily a solid block. The grinding step can be followed by a polishing step, which may release the stresses induced by the grinding process. The polishing step can be performed in the same tool as the grinding step, e.g. by applying another polishing disk onto the rotating table of a rotatable grinding tool.

Instead of grinding only from the backside of the shank or probe, resulting in a pointed tip in the first side 50 of the probe or probe array, it is also possible to grind from both sides resulting in a pointed tip located in between the first and second side 50/51, and preferably at the centre of the shaft's thickness. This can be done by grinding the probe in two steps: in the first step, the probe is attached by its fin 30 (not shown) to the carrier block 110 as described above and a portion of the tip is removed by grinding and possibly polishing, preferably until at least about half of the straight edge 100 is removed. Then the probe is removed, reversed and re-attached by its first side 50, and a second grinding and possibly polishing step is done until a sharp tip is obtained, the tip lying in between the first and second side 50/51 of the probe. Both steps can be done by the method of the disclosure according to the embodiment of FIG. 14 or FIG. 15.

The sharpness of the carrier's edge 113 is not a crucial parameter. It is possible to grind the tip of the probe while the carrier contains a blunt edge 113. Since the angle of the carrier 110 and the thickness of the tip are known, one can calculate how far the chisel shaped tip at the distal end should extend from the edge 113.

Referring to FIGS. 2A-2G and 3A-3G, in a first step, an insulating layer 14, 11 is deposited on both wafer sides 2, 3. The layer 14, 11 is a stress compensated stack of a thermal silicon dioxide (SiO2) grown at 950° C., a silicon nitride (Si3N4) produced by low-pressure chemical vapor deposition (LPCVD) at 770° C. and a LPCVD low temperature oxide (LTO) deposited at 425° C. The corresponding thicknesses are 200 nm, 100 nm and 200 nm. The stress compensation of the different layer stacks is based on residual stress values.

The metallization of electrode 4 consists of 30 nm titanium (Ti), 200 nm gold (Au), 100 nm platinum (Pt) and 30 nm Ti. These layers are deposited by evaporation and patterned using a lift-off process. The evaporation of the metals was performed in a static deposition mode with the wafer position fixed above the evaporation source. By this choice frayed metal edges are avoided. In contrast, in the alternative dynamic mode, where up to six wafers are rotated above crucibles, negatively tapered resist sidewalls are partly covered due to an increased angle of incidence. While titanium serves as the adhesion layer to the dielectric thin films, gold is used to minimize the interconnection resistance and platinum is the electrode material of choice. The metal stack is covered by a 1.5 micro meters (μm) thick passivation multilayer 5. This is again a stress compensated layer stack of alternating PECVD SiOx (210 nanometers (nm)) and SixNy (160 nm) thin films. The passivation is patterned by reactive ion etching (RIE) using the STS RIE Multiplex etcher of Surface Technology Systems, UK, thus opening electrodes and contact pads, as illustrated in FIGS. 2A and 3A. A RIE recipe optimized for the etching of SiO2 with a platen power of 219 W; a process pressure of 100 millitorr (mTorr); and gas flows of 15 standard cubic centimeters per minute (sccm), 35 sccm and 50 sccm, for tetrafluoromethane (CF4), fluoroform (CHF3) and argon, respectively, is applied. As the dry etching stops at the Ti surface, Pt is protected against the CF4/CHF3 plasma.

A subsequent wet etching of the upper Ti on the electrodes and contact pads in 1% hydrofluoric acid (HF) exposes the Pt electrodes.

The rear SiOx/SixNy etch masks 11 are patterned using RIE, as shown in FIGS. 2B and 3B. A photoresist layer 10 is spin coated on the rear of the wafer to define the step of the T-shape, as shown in FIG. 2C. The first DRIE step using an STS ICP Multiplex etcher from Surface Technology Systems, UK etches the T-shape step, as shown in FIG. 2D. Next, the photoresist 10 is removed, as shown in FIGS. 2E and 3E, and the second DRIE 9 is applied, which defines the overall geometry of the shaft thickness (also using an STS ICP Multiplex Etcher from Surface Technology Systems, UK), see FIGS. 2F and 3F.

The final DRIE 9 is performed from the wafer front prior front side patterning of the SiOx/SixNy passivation layer 5 (using RIE) to complete the release of the probes, as shown in FIGS. 2G and 3G. The fabrication wafer with probes suspended by a pair of thin struts is finally separated from the support wafer.

It is to be understood that the disclosure is not limited to the particular features of the means and/or the process steps of the methods described as such means and methods may vary. It is also to be understood that the terminology used herein is for purposes of describing particular embodiments only, and is not intended to be limiting. It is also to be understood that plural forms include singular and/or plural referents unless the context clearly dictates otherwise. It is moreover to be understood that, in case parameter ranges are given which are delimited by numeric values, the ranges are deemed to include these limitation values.

The particular combinations of elements and features in the above detailed embodiments are exemplary only. As those skilled in the art will recognize, variations, modifications, and other implementations of what is described herein can occur to those of ordinary skill in the art without departing from the spirit and the scope of the disclosure as claimed. Accordingly, the foregoing description is by way of example only and is not intended as limiting. The disclosure's scope is defined in the following claims and the equivalents thereto. Furthermore, reference signs used in the description and claims do not limit the scope of the disclosure as claimed.

1.-17. (canceled)

18. A probe for recording and/or stimulating activity in a brain, comprising:
a connecting portion; and
at least one shank extending from the connecting portion, wherein the at least one shank includes a first side, the first side including at least one recording and/or stimulating site, a second side opposed to the first side, and a fin protruding substantially perpendicularly from the second side and running on at least a part of a length of the at least one shank.

19. The probe according to claim 18, wherein the at least one shank includes a T-shaped cross-section provided by the fin protruding substantially perpendicularly from the second side.

20. The probe according to claim 18, wherein the first side has a substantially constant orientation along the length of the at least one shank, and the second side includes an angled profile along the length of the at least one shank.

21. The probe according to claim 18, wherein the at least one shank includes a triangular shaped tip at a distal end from the connecting portion.

22. The probe according to claim 18, wherein the at least one shank includes a chisel shaped tip at a distal end from the connecting portion.

23. The probe according to claim 18, wherein the at least one shank includes a sharp pointed tip at a distal end from the connecting portion, the sharp pointed tip formed in a plane defined by the first side.

24. A probe for recording and/or stimulating activity in a brain, comprising:
a connecting portion;
a first shank extending from the connecting portion and including a first side, a second side opposed to the first side, and a fin protruding substantially perpendicularly from the second side and running on at least a part of a length of the first shank, wherein the first side includes at least one recording and/or stimulating site; and
a second shank extending from the connecting portion, substantially parallel to the first shank, and including a first side, a second side opposed to the first side, and a fin protruding substantially perpendicularly from the second side of the second shank and running on at least a part of a length of the second shank, wherein the first side of the second shank includes at least one recording and/or stimulating site,
wherein,
the fin of the first shank is attached to one of the fin of the second shank, the first side of the second shank, and the second side of the second shank, thereby linking the first shank to the second shank.

25. A probe for recording and/or stimulating activity in a brain, comprising:
a connecting portion;
a first set of shanks extending from the connecting portion, wherein each of the first set of shanks includes a first side, and a second side opposed to the first side, wherein the first side includes at least one recording and/or stimulating site; and
a second set of shanks extending from the connecting portion and having substantially a similar orientation as that of the first set of shanks, wherein each of the second set of shanks includes a first side, and a second side opposed to the first side, wherein the first side includes at least one recording and/or stimulating site, and wherein the second side of each of the second set of shanks is attached to the second side of one of the first set of shanks, thereby providing two sets of substantially parallel shanks with each of the first set shanks being linked to one of the second set of shanks.

26. A method for manufacturing a probe for recording and/or stimulating activity in a brain, the probe comprising a connecting portion and at least one shank, extending from the connecting portion, the method comprising:
providing a substrate having a first surface and a second surface opposed to the first surface, wherein the first surface includes at least one recording and/or stimulating site;
providing on the second surface an etching mask pattern delimiting a shape of the connecting portion and of the at least one shank, the etching mask pattern being resistant to an etching process;
performing a first etching of the provided substrate to form the connecting portion and the at least one shank, wherein the at least one shank includes a first side and a second side opposed to the first side, and wherein the first side includes at least one recording and/or stimulating site; and
providing a fin to the second side of the at least one shank, wherein the fin protrudes substantially perpendicularly from the second side and runs on at least a part of a length of the at least one shank.

27. The method according to claim 26, wherein providing the fin comprises:
performing a second etching on the formed at least one shank to form the fin.

28. The method according to claim 26, wherein providing the fin comprises:
attaching the fin to the at least one shank.

29. The method according to claim 26, further comprising:
removing at least partially the formed at least one shank and the connecting portion from the substrate; and
sharpening the at least one shank at a distal end from the connecting portion to form a sharp pointed tip, wherein the tip is formed in a plane defined by the first side.

30. The method according to claim 29, wherein sharpening the at least one shank comprises:
providing a carrier having first and second substantially flat outer surfaces, which form an angle between each other at an edge of the carrier;
securing the at least one shank to the second flat outer surface of the carrier, wherein the second side of the at least one shank faces the second flat outer surface of the carrier and the distal end is positioned in close proximity to the carrier edge, and
subjecting the first flat outer surface of the carrier and at least part of the distal end of the at least one shank probe to a grinding process, thereby removing material at least from the distal end.

31. The method according to claim 27, wherein performing the second etching on the formed at least one shank to form the fin further comprises:
providing a second etching mask pattern on the second side, wherein the second etching mask pattern is configured to mask an area corresponding to the fin of the at least one shank, the second etching mask patter being resistant to an etching process; and
etching the second side in a direction substantially perpendicular to the second side in such a way as to thin at least part of the substrate comprising the at least one shank, thereby defining a height of the fin.

32. The method according to claim 26, wherein the at least one shank comprises a T-shaped cross-section.

33. The method according to claim 26, wherein the probe further comprises a plurality of shanks, extending from the connection portion and having substantially a similar orientation.

34. The method according to claim 26, wherein the first etching mask pattern is configured to mask an area corresponding to a location of each fin of the plurality of shanks.
